(19) United States
(12) Patent Application Publication
(10) Pub. No.: US 2013/0310658 A1
(43) Pub. Date: Nov. 21, 2013

(54) Activity Measurement Systems

(71) Applicants: Nathan W. Ricks, Lehi, UT (US); Dustin Bybee, Lehi, UT (US); Caleb Manscill, Lehi, UT (US)

(72) Inventors: Nathan W. Ricks, Lehi, UT (US); Dustin Bybee, Lehi, UT (US); Caleb Manscill, Lehi, UT (US)

(21) Appl. No.: 13/871,934

(22) Filed: Apr. 26, 2013

Related U.S. Application Data
(60) Provisional application No. 61/638,909, filed on Apr. 26, 2012.

(51) Int. Cl. A61B 5/11

(52) U.S. Cl. CPC A61B 5/1118
USPC 600/301; 600/409; 600/549; 600/595; 600/483

(57) ABSTRACT

A system for monitoring the health and activity of an individual is disclosed. The system includes a mobile communication device and a body attachment, such as an armband. The body attachment has one or more of an accelerometer, altimeter, temperature sensor, and/or magnetometer.

### CROSS-REFERENCE TO RELATED APPLICATIONS

This application claims benefit to U.S. Provisional Patent Application Ser. No. 61/638,909 filed Apr. 26, 2012.

### BACKGROUND

1. Field of the Invention

The present invention relates generally to the measuring physical activity and health conditions of an individual. More specifically, the invention relates to an activity measurement system that includes an armband or other such body attachment configured to measuring various aspects of an individual's personal activity and health and communicate these measurements to a mobile communication device. Specifically, some implementations of the activity measurement systems measure one or more of an individual's movement, body temperature, body movement, heart rate, and blood pressure.

2. Background

The benefits of managing one's weight, eating properly, and maintaining an active lifestyle are well known and researched. These benefits include a low propensity to illness and disease and prolonged life expectancy. Despite these advantages, the general population is experiencing an increase in body weight and a decline in many aspects of its health. One potential cause of this trend is lack of reliable feedback available to specific individuals about various aspects of their health. For instance, while many people understand the need for healthy habits, they often do not recognize the effect of their various daily lifestyle choices and unhealthy habits. Instead, other priorities can divert an individual's focus from health to other cares. As a result, well-intentioned individuals may unintentionally become heavier and less healthy.

To address these challenges, hundreds of fad diets and weight-loss plans promise quick and easy weight loss and good health. These programs try to make weight loss and good health easier, quicker, cheaper, more reliable, and less painful. While these programs can be effective and beneficial, they are often short lived and do not promote long-term health and wellness. These programs can also take an individual's focus off of the more fundamental and essential elements of health and wellness, including healthy eating habits, exercise, and physical activity.

Additionally, because many health problems develop over long periods of time, some people are unaware of the negative effects of their lifestyle choices. For instance, because many individuals feel good or are content with their overall appearance and body weight they fail to recognize or addressing their unhealthy lifestyle practices. These individuals may later experience serious health problems that they may have avoided if they had understood their effects of their lifestyle choices or if they had been advised or their potential for illness and advised to make preventative changes.

Thus, while current health programs and systems are available, improvements would be desirable.

### SUMMARY

The present invention relates generally to the measuring physical activity and health conditions of an individual to encourage healthy lifestyle practices. More specifically, the invention relates to an activity measurement system that includes an armband or other body attachment device configured to measuring various aspects of an individual's personal activity and overall health and communicate these measurements to a mobile communications device. For example, the body attachment can include accelerometer, altimeter, and/or magnetometer, which can measure an individual's three-dimensional movements. In some implementations, the body attachment can also include a heart rate monitor, body temperature sensor, and/or blood pressure monitor to measure the individual's body characteristics. These measured characteristics can be used to calculate the calorie expenditure of the individual.

In some implementations, the body attachment can communicate these measured values to a mobile communication device, such as a smart phone, which can process these measurements using one or more modules and display them on a display screen. The mobile communication device can employ a mobile application (“app”) configured to present the user with information about the distance traveled, steps taken, activity duration, calories burned, and/or body temperature. The mobile application can also provide various other forms of health-related information, tools, logs, charts, advice, and warnings. In some instances, the network device can be configured to relay the health measurements to a health professional, health insurance organization, physical trainer, or other third party.

The measurement and presentation of the user's activity measurements and health conditions can provide the user with information and tools that can assist the user to manage his/her weight and overall health. This system can thus avoid the downsides of temporary health fads by offering actual, measurable information that can be used to form enduring, effective, and improved lifestyle changes. This information can empower the users to managing their weight, eat properly, and maintaining an active lifestyle in order to reduce their propensity to illness and disease and prolong their life expectancy.

These and other features and advantages of the present invention may be incorporated into certain embodiments of the invention and will become more fully apparent from the following description and appended claims, or may be learned by the practice of the invention as set forth hereinafter. The present invention does not require that all the advantageous features and all the advantages described herein be incorporated into every embodiment of the invention.

### BRIEF DESCRIPTION OF THE DRAWINGS

In order that the manner in which the above recited and other features and advantages of the present invention are obtained, a more particular description of the invention will be rendered by reference to specific embodiments thereof, which are illustrated in the appended drawings. Understanding that the drawings depict only typical embodiments of the present invention and are not, therefore, to be considered as limiting the scope of the invention, the present invention will be described and explained with additional specificity and detail through the use of the accompanying drawings.

FIG. 1 illustrates a representative activity measurement system, according to some embodiments of the present invention.

### DETAILED DESCRIPTION OF THE INVENTION

A description of embodiments of the present invention will now be given with reference to the Figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

The following disclosure of the present invention is grouped into subheadings. The utilization of the subheadings is for convenience of the reader only and is not to be construed as limiting in any sense.

For the purposes of the present invention, the phrase “A/B” means A or B. For the purposes of the present invention, the phrase “A and/or B” means “(A), (B), or (A and B).” For the purposes of the present invention, the phrase “at least one of A, B, and C” means “(A), (B), (C), (A and B), (A and C), (B and C), or (A, B and C).”

Various operations may be described as multiple discrete operations in turn, in a manner that may be helpful in understanding embodiments of the present invention; however, the order of description should not be construed to imply that these operations are order dependent.

The description may use the phrases “in an embodiment,” or “in various embodiments,” “in some configurations,” or “in some instances,” which may each refer to one or more of the same or different embodiments. Furthermore, the terms “comprising,” “including,” “having,” and the like, as used with respect to embodiments of the present invention, are synonymous with the definition afforded the term “comprising.”

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

The present invention relates generally to measuring a user's physical activity and health conditions. More specifically, the invention relates to an activity measurement system, such as an armband or other such body attachment, configured to measuring various aspects of an individual's personal activity and overall health.

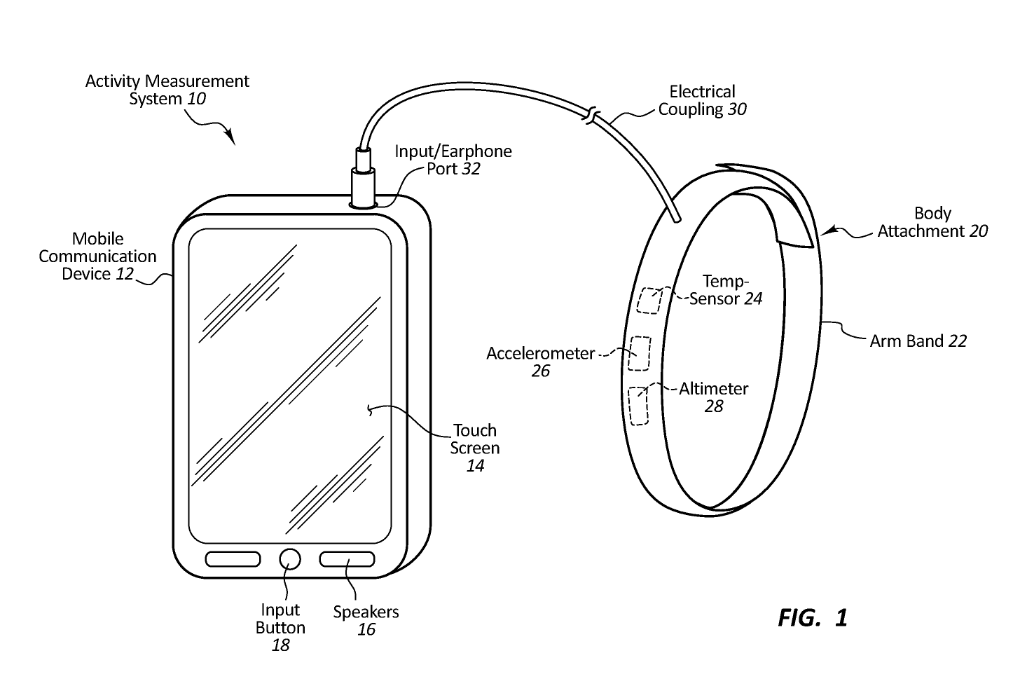

Reference will now be made to FIG. 1, which illustrates a representative activity measurement system 10, according to some embodiments. The activity measurement system 10 can generally includes a mobile communication device 12 and a body attachment 20. The body attachment 20 can be worn or carried by a user. The body attachment 20 can take various measurements and transmit this information to the mobile communication device 12. The mobile communication device 12 can then process these measurements to identify and measure the user's physical activities, movements, heart rate, blood pressure, temperature, calories burned, and or other measurements. The mobile communication device 12 can then display this information to the user on a display screen (which can be a touch screen) 14, via a mobile application or other suitable software program.

The measurement and presentation of the user's health measurements and information can provide the user with information and tools that can assist the user to manage his/her weight and overall health. This system can avoid the downsides of temporary health fads by offering actual, measurable information that can be used to form enduring, effective, and improved lifestyle changes. This information can empower the users to managing their weight, eat properly, and maintaining an active lifestyle in order to reduce their propensity to illness and disease and prolong their life expectancy.

Each of the respective components of the activity measurement system 10 will be described in detail in the sections presented below.

### Body Attachment

Reference will continue to be made to FIG. 1 and particularly to the body attachment 20. The body attachment 20 can be an anklet, bracelet, clip, finger ring, toe ring, pendant, necklace, earring or other piercing, implant 22, or other attachable or carry-able device that takes one or more activity measurements. These activity measurements can be taken using one or more sensors and other devices and systems. Particularly, the body attachment 20 may be used to acquire activity measurements that are can be used to track user's physical activity levels and performance.

Specifically, the body attachment 20 can include one or more sensors or measurement devices for measuring the user's activity and health levels. For example, in some embodiments, the body attachment 20 can have an accelerometer 26, altimeter 28, temperature sensor 24, magnetometer (not shown), heart rate monitor (not shown), blood pressure monitor (not shown) and/or various other sensors and measurement devices. Additionally, or alternatively the body attachment can include one or more pedometer, tilt sensor, and/or global positioning system (GPS) device. The accelerometer 26, altimeter 28, magnetometer, pedometer, tilt sensor, and/or GPS device can be used to count the steps the wearer takes, measure the altitudes the wearer ascends and descends, recognize the direction the wearer is facing and advancing, recognize the speed a wearer is traveling at, and identify up and down movements such as jumping or bouncing. These devices can thus be used separately or in combination to measure the wearer's three-dimensional movement and activities.

The body attachment 20 can additionally or alternatively include one or more sensors or measurement device configured to measure various body conditions of the user. For example, the body attachment 20 can include a temperature sensor 24 for measuring the user's body temperature. Body temperature measurements can be used to determine the physical activity level of the users, since physical activity can generate body heat. In some configurations, the temperature sensor 24 can include one more electrodes or other contact surface that can be placed in contact with the user's skin. The one or more electrodes can include a thermocouple, thermister, or other temperature sensor to detect temperature levels of the user's skin surface.

In some embodiments, the body attachment 20 includes a heart rate monitor. Heart rate is the number of heartbeats per unit of time (e.g., a minute). Heart rate measurements can assist to determine the level of health of the user and to diagnose and track medical conditions. For instance, healthy or active individuals may have a lower resting heart rate than less active or less healthy individuals. Accordingly, in some embodiments, the body attachment 20 can include an electrocardiogram (ECG or EKG) or other device for measuring the user's heart rate.

In some embodiments, the activity measuring system 10 can further includes one or more devices for measuring the user's blood pressure. Blood pressure one of the principle vital signs and can provide useful health information to the user and health professionals. Blood pressure is the pressure that blood exerts upon blood vessel walls within the body. During a heartbeat, blood pressure rises from a diastolic pressure to a systolic pressure, each of which can be measured and tracked by the health measurement system. Healthy users can have lower diastolic and systolic blood pressure values. By providing blood pressure measurements to a user, the user can gauge his/her stress levels and progress in maintaining healthy blood pressure levels. In some embodiments, the activity measuring system can measure the user's blood pressure using a pulse oximeter or other suitable device. The pulse oximeter can include one or more light sources (e.g. light-emitting diodes) that transmit light into the skin of a patient. The pulse oximeter can also include one or more detectors, such as a photo detector that detects light reflected from an underlying artery. By processing the character of the reflected and the transmitted lights, the pulse oximeter can identify and measure various features of the user's blood, including the user's blood pressure, and oxygen saturation.

Some embodiments of the body attachment 20 can include one or more devices for measuring the user's blood glucose levels. Blood glucose levels, which indicate the amount of glucose in the blood, can be important information for users with diabetes. For instance, in some configurations, the activity measuring system can incorporate an external glucometer that can interface and communicate with the activity measuring system. For example, an external glucometer can communicate with one or more components of the activity measuring system via a short-range wireless link (e.g., a Bluetooth link). Blood glucose levels can be received, stored, and/or transmitted by the body scale when the external glucometer is located within a minimum range in which the wireless link can be established.

Various embodiments of the body attachment can include one or more internal or external sensors or other such devices for measuring the user's C-reactive protein levels. C-reactive protein is found the blood and can indicate a rise in inflammation and infection. High levels of C-reactive protein can be problematic to users with heart related diseases. Accordingly, it can be advantageous to monitor the levels of this protein in the blood. In some instances, an external C-reactive protein sensor can communicate with the mobile communication device 12 via a short-range wireless link.

In some embodiments, the body attachment 20 can also incorporates one or more additional internal or external sensors that can be used to measure ketone levels within the blood. Ketone bodies include water-soluble compounds that are produced in the liver and kidneys. Ketosis occurs when the blood accumulates an excessive level of ketone bodies. This can be cause by eating disorders and low carbohydrate intake. The measurement of ketone levels can assist the user to recognize if his/her ketone levels are in a healthy range or if ketosis is taking place.

In some embodiments, the activity measurement system 10 incorporate one or more additional internal or external sensors that can be used to measure blood cholesterol levels, testosterone levels within the blood, progesterone levels within the blood, and/or drug levels within the blood. These measurements can be useful in providing health and fitness related information to the user and for determining the overall health of the user.

It will be understood that the body attachment 20 can include other devices used to measure other characteristics and conditions not described herein.

The measurements taken by the body attachment 20 can be transmitted to the mobile communication device via a communication link. This communication link can be a wireless or direct-wired communication link. For example, in some configurations, this communication link is a short-range wireless link (e.g., a Bluetooth link). As such, the body attachment 20 can upload its health measurements to the mobile communication device 12 when the body attachment 20 is in proximity to the mobile communication device 12, such as within 15 feet. Such wireless communication can enable easy and rapid data transmission. In other configurations, this second communication link can be a wired communication link, such as the cable 30 shown in FIG. 1. The wired communication link can be coupled to an earphone port 32 or other port of the mobile communication device 12. A wired connection can advantageously charge any batteries within the body attachment 20.

In some embodiments, the activity measuring system 10 incorporates the components of the body attachments into the mobile communication device 12 so that a body attachment 20 is not needed or used. In these embodiments, the mobile communications device 12 can contain or include the activity and health condition sensors and measurement devices previously mentioned. For instance, the mobile communications device 12 can include one or more accelerometer 26, altimeter 28, magnetometer, pedometer, tilt sensor, GPS device, and/or sensors or measurement device to measure various body conditions. In use, the mobile communication device 12 can be strapped to the skin of a user, or otherwise positioned to take the various measurements.

### Mobile Application

Referring still to FIG. 1, after the mobile communication device 12 has received the transmitted measurements from the body attachment 20, these measurements can process, analyzed, and display on a display screen 14. The mobile communication device 12 can employ a mobile application (“app”) configured to present the wearer with information about the distance traveled, steps taken, activity duration, calories burned, body temperature, and other measured or calculated health conditions. The mobile application can also provide various other forms of health-related information, tools, logs, charts, advice, and warnings.

In some configurations, the mobile communication device 12 is configured to relay measurements from the body attachment 20 to an external computer system over a wireless communication network. This network can include a cellular network employing a 3G, GSM, or CDMA communication standard. The external computer system can process these measurements and provide processed measurements and other information to the user via the mobile application and/or a separate website.

In other configurations the mobile communication device 12 or mobile application can include one or more modules configured to calculate the calories consumed by the wearer during a period of time, such as during an entire day or during a workout. This calculation can also be continuously updated during the entire period of use of the body attachment 20. In some embodiments, the one or more software modules can utilize the body temperature measurements, heart rate measurements, and/or activity measurements to calculate or approximate the user's calorie consumption.

Additionally, the mobile application can use the activity measurements to provide information about the user's sleep habits. For example, by identifying when a user (wearing the body attachment 20) stops or slows his/her movements, the activity measurement system 10 can identify when the user goes to bed, when the user falls asleep, how often the user wakes up during his/her sleep, and when the user wakes up. By analyzing the information, the activity measurement system 10 can identify the duration of the user's sleep and the overall quality of the sleep. By combining such sleep measurements from a single day with measurements over a period, the activity measurement system 10 can identify the user's sleep behaviors, patterns, or lack of patterns and provide analysis, information, and sleep suggestions to the user.

In some embodiments, the mobile application can also use the user's activity measurements to track a user's exercise activities, including when a user exercises and the duration of the exercise. This information can be used to recognize if the user is adhering to an exercise program. This information can be automatically charted for the user. This information can also be used to transmit reminders, motivational messages, and congratulations to the user.

In addition to calculating and presenting information, the activity measuring system 10 can also identify potential health risks or health conditions identified by analyzing the activity measurements and measured health conditions. For example, using the measured heart rates, the activity measuring system 10 can identify any present heart rate abnormalities, such as arrhythmia, bradycardia, or atrial fibrillation by monitoring the heart rate and regularity. In addition, the activity measuring system 10 can advise the user to slow down if the user's heart rate is too high, or if the user is prone to injury by exercising too frequently during the initial days and weeks of an exercise program.

In some embodiments, the mobile application can receive input information, such as the user's age, height, health history, fitness goals, and health goals. In some instances, the website or mobile application can provide an interface for the use to submit the type and amount of food that the user consumed in a day, in order to calculate the user's calorie intake. After calculating calorie intake, the mobile application can use the calories burned measurements to calculate the net calorie gain or loss. This information can be tracked by a user who is trying to gain or lose weight.

In additional to presenting activity measurements to the user, the mobile application can provide various other forms of health related information, tools, logs, charts, advice, and warnings. For example, the mobile application can chart the user's activity history, exercise history, weight, body fat percentage, resting heart rate, or blood pressure over a period, such as a month or year. In another example, the mobile application can chart the user's heart rate, body temperature, and/or calorie expenditure during a period of exercise. In yet another example, the mobile application can chart the user's sleep measurements during a time while providing analysis and suggestions for improved sleep practices. In still another example, the mobile application can provide an overview of the user's health, including warning, areas of cautions, and general tips. The website or mobile application can also provide one or more interfaces for recording a user's goals, charting the user's progress, and providing goal related messages, reminders, and encouragement.

In some instances, the activity measuring system 10 can be configured to relay the activity measurements to a health professional, health insurance organization, physical trainer, or other third party. These individuals may be granted access to the user's activity measurements and related information. Using this information, these third parties can track a user's general and specific activity levels and provide feedback or analysis. For instance, a physical trainer can receive periodic updates of the user's weight or body fat percentage and provide exercise program modifications and updates. Furthermore, it will be understood that various other types of tools, features, and information can be provided to the user via the mobile application.

### Mobile Communication Device

Reference will now be made specifically to the mobile communication device 12 of FIG. 1. The mobile communication device 12 can be a hand held electronics device, such as a smart phone, tablet computer, personal digital assistant, or other similar device. As shown, the mobile communication device 12 can include a touch screen, audio port 32, speakers 16, and/or an input button. Non-limiting examples of mobile communications devices include an iPhone or iPad from Apple, Inc., a Galaxy from Samsung, Inc., or a Droid from Motorola. One skilled in the art will appreciate that embodiments of the invention may be practiced by various types and models of mobile communication computing and 12.

Embodiments of the mobile communication device 12 can embrace one or more computer-readable media, wherein each medium may be configured to include or includes thereon data or computer executable instructions for manipulating data. The computer executable instructions include data structures, objects, programs, routines, or other program modules that may be accessed by a processing system, such as one associated with a mobile communication device 12 capable of performing various different functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer-readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system. While embodiments of the invention embrace the use of all types of computer-readable media, certain embodiments as recited in the claims may be limited to the use of tangible, non-transitory computer-readable media, and the phrases “tangible computer-readable medium” and “non-transitory computer-readable medium” (or plural variations) used herein are intended to exclude transitory propagating signals per se.

From the foregoing, it will be seen that the measurement and presentation of the user's activity and health condition measurements can provide the user with the tools to manage his/her weight and overall health. This system can thus avoid the downsides of temporary health fads by offering actual, measurable information that can be used to form enduring, effective, and improved lifestyle changes. This information can empower the user to managing his/her weight, eat properly, and maintaining an active lifestyle in order to reduce his/her propensity to illness and disease and prolong his/her life expectancy.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A system for monitoring the health and activity of an individual, the system comprising:
a mobile communication device; and
a body attachment in electronic communication with the mobile communication device, the body attachment having one or more of an accelerometer, altimeter, temperature sensor, and/or magnetometer, the body attachment being configured to transmit measurements from the one or more of an accelerometer, altimeter, temperature sensor, and/or magnetometer to the mobile communication device.

2. The system of claim 1, wherein the body attachment further have one or more sensors configured to measure one or more of a wearer's blood pressure and heart rate.

3. The system of claim 1, wherein the mobile communication device includes a mobile application having one or more modules configured to calculate the calories used by the wearer.

4. The system of claim 1, wherein the body attachment is a ring.

5. The system of claim 1, wherein the body attachment is a watch.

6. The system of claim 1, wherein the body attachment is a band.

7. The system of claim 1, wherein the body attachment is an anklet.

8. The system of claim 1, wherein the body attachment is an implanted disposed below the epidermis of a user.

9. The system of claim 1, wherein the body attachment is in electronic communication with the mobile communication device using a wired connection.

10. The system of claim 9, wherein the wired connection includes an audio port of the mobile communication device.

11. The system of claim 1, wherein the body attachment is in electronic communication with the mobile communication device using a wireless connection.

12. The system of claim 11, wherein the wireless connection is a Bluetooth connection.

13. The system of claim 12, wherein the mobile communication device is a smart phone.

14. A system for monitoring the health and activity of an individual, the system comprising a mobile communication device having one or more accelerometer, altimeter, temperature sensor, and/or magnetometer.

15. The system of claim 14, wherein the body attachment further have at least one or heart rate sensor and a blood pressure sensor.

16. The system of claim 14, wherein the mobile communication device includes a mobile application having one or more modules configured to calculate the calories used by the person carrying the mobile communication device.

17. The system of claim 14, wherein the mobile communication device is a smart phone.
